(19) United States
(12) Patent Application Publication
(10) Pub. No.: US 2019/0275285 A1
(43) Pub. Date: Sep. 12, 2019

(54) SOOTHING SENSORY SQUARE

(71) Applicant: Drew Delp, Virginia Beach, VA (US)

(72) Inventor: Drew Delp, Virginia Beach, VA (US)

(21) Appl. No.: 15/915,951

(22) Filed: Mar. 8, 2018

(51) Int. Cl. A61M 21/02

(52) U.S. Cl. CPC A61M 21/02; A61M 2021/0044

(57) ABSTRACT

A therapeutic devices targeted towards children and adults with developmental conditions. The present invention invention provides for a sensory device that provides visual and tactile stimulation to individuals who have autism and related conditions. The device has a shape and size suited to being hand-held by a user. The device contains within the body of the device channels containing spheres that move within the channels, providing sensory and visual stimulation.

### FIELD OF THE INVENTION

The invention relates to toys and therapeutic devices targeted towards children and adults with developmental conditions. More specifically, the invention provides for a sensory device that provides visual and tactile stimulation to individuals who have autism and related conditions.

### BACKGROUND OF THE INVENTION

Providing a stimulating educational environment for individuals having developmental conditions can be difficult. Autism or autism spectrum disorder, causes impaired communication and social interaction. It can also cause obsessive and repetitive behaviors. Autistic individuals can have periods where they are extremely upset (commonly referred to as a meltdown), with common triggers being changes or disruptions in their normal routine, overstimulation, frustration, and communication difficulties. Distracting an autistic individual is a common and effective tool for helping calm them and ease the anxieties associated with the meltdown. While this disclosure discusses the new device in the context of autism, it will be understood that the device can be used for a broad range of individuals with varying developmental conditions.

Numerous devices, including toys, exist that attempt to help distract and calm an autistic individual in a meltdown. Critical to these toys are tactile, auditory, and visual features that serve to capture the attention of the individual. For example, squeeze toys may provide an item for an individual in meltdown to grasp. However, the options for visual and auditory are limited in such toys. The same is true with balls and other devices that have textured or bumpy surfaces. Visual devices include such things as moving lights, LED devices, disco balls, and the like. These devices are also limited in that they rarely provide any tactile stimulation. The same is true of auditory devices, such as noisemakers or headphones. And with visual stimulation, it is difficult to create complex and changing imagery that will keep an autistic individual interested.

As a result of these limitations, none of the existing devices, manage to work effectively across a broad range of autistic individuals. Each individual is different, with different needs, and may require a different form of stimulus than another.

What is needed, therefore, is a device that incorporates tactile, auditory, and visual stimulation that overcomes the limitations of existing devices. The device should provide all three forms of stimulation. It should also incorporate complex visual stimulation that is interesting as well as changing so that an autistic individual will continue to be interested in the device.

It is therefore an object of the present invention to provide an improved device used in the education and development of individuals with developmental disorders.

It is a further object of the present invention to provide an improved device used in the education and development of individuals with development disorders, the improved device including tactile, auditory, and visual stimulation.

It is a further object of the present invention to provide an improved device used in the education and development of individuals with development disorders, the improved device provide complex and changing visual stimulation.

### BRIEF DESCRIPTION OF THE DRAWINGS

These and other embodiments are described by the following figures and detailed description.

FIG. 1 is a view of a device in accordance with an embodiment of the present invention.

FIG. 2 is an interior structural detail of an embodiment of the present invention.

FIG. 3 is an oblique view of an embodiment of the present invention.

FIG. 4 is a view of a device in accordance with an embodiment of the present invention.

### DETAILED DESCRIPTION OF THE INVENTION

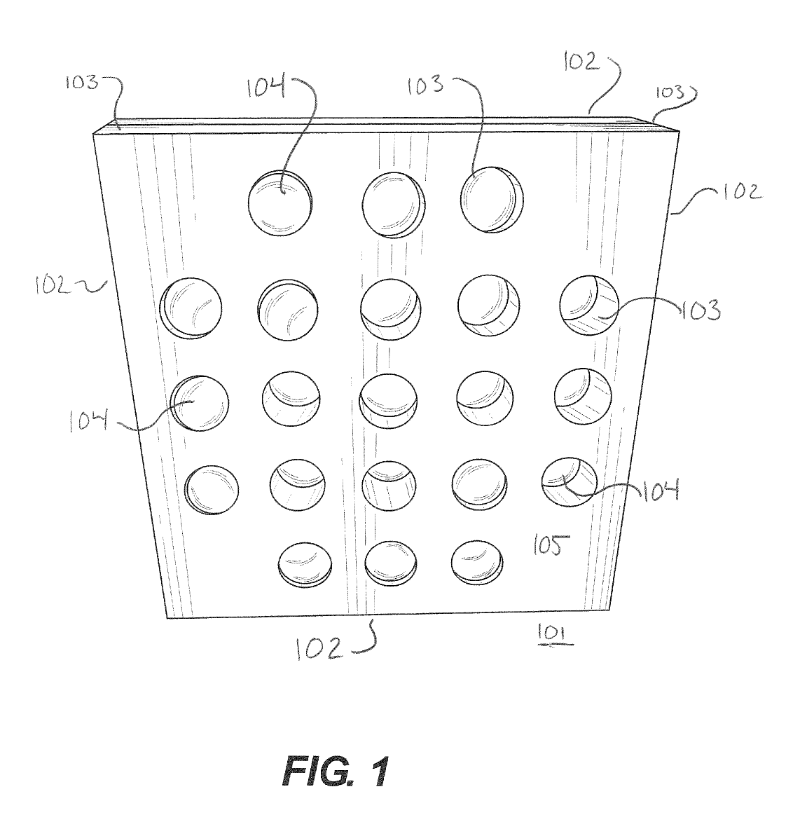

Referring now to FIG. 1, a view of a device in accordance with an embodiment of this invention is shown. The sensory device 101 is generally a solid in shape. As shown, the device 101 is a cuboid, with a first face 105 and a second face, the first and second faces being equal. The device 101 as shown further has four sides 102 with the depth of the device 101 being determined by the width of the sides 102 between the first face 105 and the second face. As will be apparent from this description, various shapes may be utilized for the device 101 without deviating from the scope and intent of the invention.

Disposed within the device 101 are channels 103, the channels 103 being generally tubular in shape. Contained within the channels 103 are generally spherical inserts 104. The inserts 104 are chosen of a size such that the diameter of the inserts 104 is slightly smaller than the diameter of the channels 103, allowing the inserts 104 to move back and forth within the channels 103, providing movement and thereby visual stimulation to the user. The inserts 104 may be of various materials. In a preferred embodiment, the inserts 104 are formed from a transparent or semi-transparent material such that light may partially or completely pass through the inserts 104, being distorted in the process. This movement and distortion creates visual stimulation for a user. The inserts 104 may be constructed of materials that are any possible color and design. A preferred embodiment utilizes clear inserts 104, but it will be understood that various colors may be utilized without deviating from the scope of the invention. The device 101 may be constructed of any rigid or semi-rigid material that may be shaped and/or constructed to accommodate the channels 103 and the inserts 104.

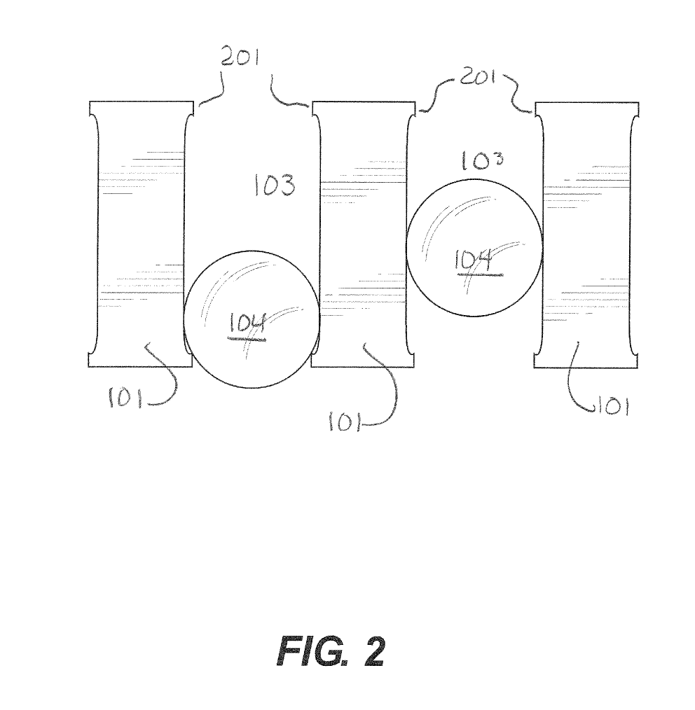

Referring now to FIG. 2, a cutaway is shown of an embodiment of the present invention. The device 101 contains channels 103, in which the inserts 104 are disposed. The inserts 104 are free to move back and forth within the channel 103 as described above. To retain the inserts 104 within the channels 103, the channels have lips 201 that are formed from the body of the device 101. The lips 102 are larger than the diameter of the channels 103, thereby allowing the inserts 104 to move freely within the channels 103 without falling out.

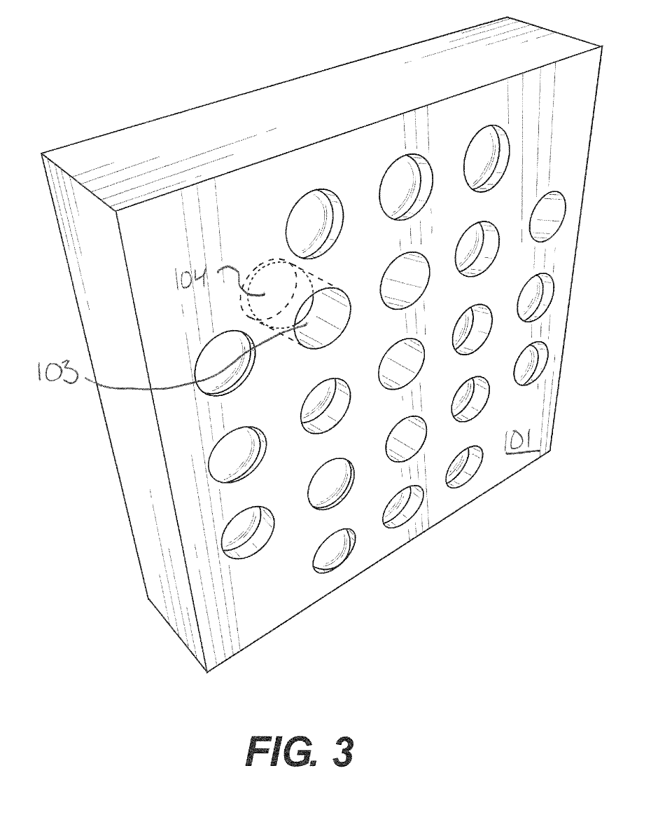

FIG. 3 shows an oblique view of an embodiment of the present invention with the channels 103 and the inserts 104 shown as disposed within the body of the device 101.

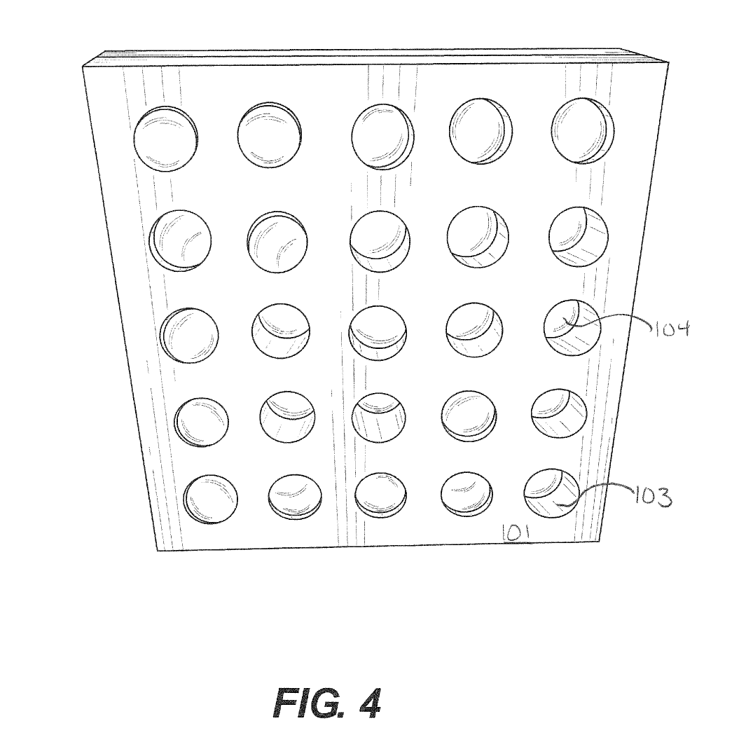

FIG. 4 shows an oblique view of an embodiment of the present invention with additional channels 103 disposed within the device 101, and inserts 104 disposed within the channels 103.

These embodiments have been described in sufficient detail to enable those skilled in the art to practice them, and it is to be understood that changes may be made without deviating from the scope of the invention described herein. It will also be understood that, although specific embodiments are shown herein, the body of the device may be of any shape chosen that provides acceptable tactile and visual sensations for a user.

What is claimed is:

1. A sensory device that provides visual and tactile stimulation to individuals who have autism and related conditions, the sensory device comprising:
A solid shape;
Disposed within the solid shape channels, the channels having lips at the openings of the channels; and
Disposed within the channels are inserts, the inserts being of a smaller diameter than the channels and the inserts being capable of movement within the channels.

2. The device of claim 1, wherein the inserts are chosen from a group of materials that are transparent or semi-transparent.

3. The device of claim 2, wherein the inserts are chosen from a group of materials that contains clear and other colors available in the group of materials that are transparent or semi-transparent.
